(19) United States
(12) Patent Application Publication
(10) Pub. No.: US 2018/0003694 A1
(43) Pub. Date: Jan. 4, 2018

(54) ISOLATED WET CELL

(71) Applicant: International Business Machines Corporation, Armonk, NY (US)

(72) Inventors: John A. Ott, GREENWOOD LAKE, NY (US); Michael A. Pereira, MOHEGAN LAKE, NY (US); Deqiang Wang, LIVINGSTON, NJ (US)

(21) Appl. No.: 15/198,698

(22) Filed: Jun. 30, 2016

(51) Int. Cl. G01N 33/487; G01Q 10/00; C12Q 1/68

(52) U.S. Cl. CPC G01N 33/48721; C12Q 1/6869; G01Q 10/00

(57) ABSTRACT

A wet cell apparatus is provided and includes a sensor body defining a nano-pore by which respective cell interiors are fluidly communicative and a scanning probe microscope (SPM) tip. The SPM tip is configured to draw a molecule through the nano-pore from one of the respective cell interiors whereby sensing components of the sensor body identify molecule components as the molecule passes through the nano-pore.

### BACKGROUND

The present invention relates to an isolated wet cell and, more specifically, to an isolated wet cell for use with a scanning probe microscope (SPM).

Deoxyribonucleic acid (DNA) sequencing refers to the process of determining the precise order of nucleotides within a DNA molecule. DNA sequencing can generally include any method or technology that is used to determine the order of the four bases (i.e., adenine, guanine, cytosine and thymine) in a strand of DNA. The advent of rapid DNA sequencing methods has greatly accelerated biological and medical research and discovery.

The resultant and increasing knowledge of DNA sequences has become indispensable for basic biological research and in numerous applied fields such as medical diagnosis, biotechnology, forensic biology, virology and biological systematics. The rapid speed of sequencing attained with modern DNA sequencing technology has been instrumental in the sequencing of complete DNA sequences, or genomes of numerous types and species of life, including the human genome and other complete DNA sequences of animal, plant and microbial species.

The methods and technologies used to perform DNA sequencing can be applied to Ribonucleic acid (RNA) and to any other macro-molecule such as a long strand of a protein whose constituent parts need to be identified and analyzed.

### SUMMARY

According to one or more embodiments of the present invention, a wet cell apparatus is provided and includes a sensor body defining a nano-pore by which respective cell interiors are fluidly communicative and a scanning probe microscope (SPM) tip. The SPM tip is configured to draw a molecule through the nano-pore from one of the respective cell interiors whereby sensing components of the sensor body identify molecule components as the molecule passes through the nano-pore.

According to one or more embodiments, a wet cell apparatus is provided and includes lower and upper cells, a sensor body formed to define a nano-pore disposed to permit fluid communication between respective interiors of the lower and upper cells and a scanning probe microscope (SPM) tip. The SPM tip is configured to draw a molecule from the interior of the lower cell into the interior of the upper cell through the nano-pore. The sensor body includes sensing components configured to identify components of the molecule as the molecule passes through the nano-pore.

According to one or more embodiments, a method of operating a wet cell apparatus is provided. The wet cell apparatus includes lower and upper cells disposed on either side of a sensor body that includes sensing components and defines a nano-pore with respective interiors of the lower and upper cells charged with a fluid and the interior of the lower cell initially charged with molecules. The method includes controlling a scanning probe microscope (SPM) tip to draw at least one of the molecules from the interior of the lower cell into the interior of the upper cell through the nano-pore and identifying components of the molecule as the molecule passes through the nano-pore and interacts with the sensing components.

### BRIEF DESCRIPTION OF THE DRAWINGS

The subject matter which is regarded as the invention is particularly pointed out and distinctly claimed in the claims at the conclusion of the specification. The forgoing and other features, and advantages of the invention are apparent from the following detailed description taken in conjunction with the accompanying drawings in which:

FIG. 1 is a side view of a well cell apparatus in accordance with embodiments;

FIG. 2 is an enlarged side view of a sensor body of the wet cell apparatus of FIG. 1;

FIG. 3 is an enlarged top down view of a lower layer of the sensor body of FIG. 2;

FIG. 4 is an enlarged top down view of an upper layer of the sensor body of FIG. 2

FIG. 5 is an enlarged top down view of an intermediate layer of the sensor body of FIG. 2

FIG. 6 is a schematic view of an SPM apparatus in accordance with embodiments;

FIG. 7 is a side view of a tapered tip of an SPM tip penetrating into a nano-pore;

FIG. 8 is a side view of a tapered tip of an SPM tip drawing a strand of DNA through a nano-pore;

FIG. 9 is a side view of a wet cell apparatus in accordance with further embodiments; and

FIG. 10 is a flow diagram illustrating an operation of a wet cell apparatus in accordance with embodiments.

### DETAILED DESCRIPTION

Fast, accurate and cost effective sequencing of DNA, RNA or any other macroscopic molecule is a highly desirable process. Thus, as will be described below, a functionalized scanning probe microscope (SPM) tip is provided to thread a molecule of DNA or RNA or any other macroscopic molecule through a nano-pore defined between two wet cells charged with fluid. As the molecule passes through the nano-pore, sensors of electrical or chemical signatures will identify various components of the molecule. For example, electrical sensors proximate to the nano-pore will be able to identify base sequences in a given strand of DNA passing through the nano-pore.

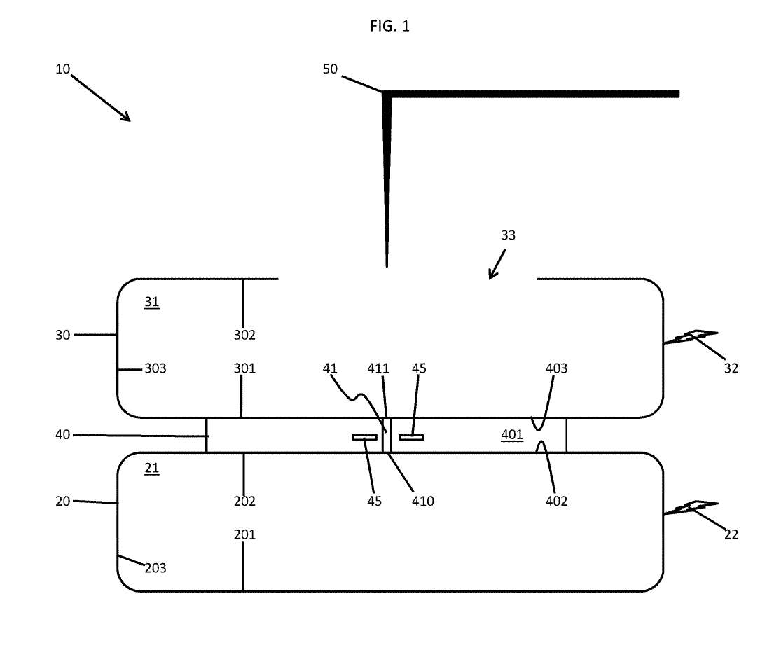

With reference to FIG. 1, a wet cell apparatus 10 is provided. The wet cell apparatus 10 includes a lower cell 20, an upper cell 30, a sensor body 40 and an SPM tip 50. The sensor body 40 is formed to define a nano-pore 41 by which an interior 21 of the lower cell 20 is fluidly communicative with an interior 31 of the upper cell 30. The SPM tip 50 is controlled and configured to draw a molecule (e.g., a strand of DNA) through the nano-pore 41 from the interior 21, through the nano-pore 41 and to the interior 31 whereby sensing components 45 of the sensor body 40 identify molecule components (e.g., base sequences of the DNA strand) as the molecule passes through the nano-pore 41.

For purposes of clarity and brevity, the following description will relate to the exemplary case where strands of DNA are drawn by the SPM tip 50 through the nano-pore 41 such that their respective base sequences can be identified. Other cases, such as the identification of components of RNA and other macroscopic molecules need not be discussed but are included within the scope of this application.

The lower cell 20 may have a generally rectangular shape with a base surface 201, an upper surface 202 and sidewalls 203 that are arranged to define the interior 21. An electrical contact 22 may be disposed in connection with any portion of the lower cell 20 in order to provide for a ground voltage or voltage bias so the strand of DNA can be manipulated (e.g., stretched or trapped) so that its dwell time in the nano-pore 41 is sufficient of sensing by the sensing components 45 of the sensor body 40. The sensor body 40 is disposable on the upper surface 202 with the nano-pore 41 aligned with a corresponding nano-aperture 410 defined in the upper surface 202.

The upper cell 30 may have a generally rectangular shape with a base surface 301, an upper surface 302 and sidewalls 303 that are arranged to define the interior 31. An electrical contact 32 may be disposed in connection with any portion of the upper cell 30 in order to provide for a ground voltage that can be used to operate the sensing components 45 of the sensor body 40. The base surface 301 is disposable on the sensor body 40 with a nano-aperture 411, which is defined in the base surface 301, aligned with the nano-pore 41. The upper surface 302 may be formed to define an upper aperture 33 by which the SPM 50 has access to at least the interior 31 and the nano-pore 41 and, in some cases, the interior 21.

Materials of the lower cell 20 and the upper cell 30 may include Lucite™ or any other similarly dielectric, generally non-volatile material. The materials may be opaque or transparent.

At least at an initial time, the interior 21 of the lower cell 20 may be charged with fluid and strands of DNA. The fluid can be any fluid, such as water or any other PH neutral or PH biased fluid. The strands of DNA may be dispersed throughout the interior 21 and may exhibit Brownian motion therein. The fluid can fill the interior 21 of the lower cell 20, the nano-pore 41 and at least a portion of the interior 31 of the upper cell 30.

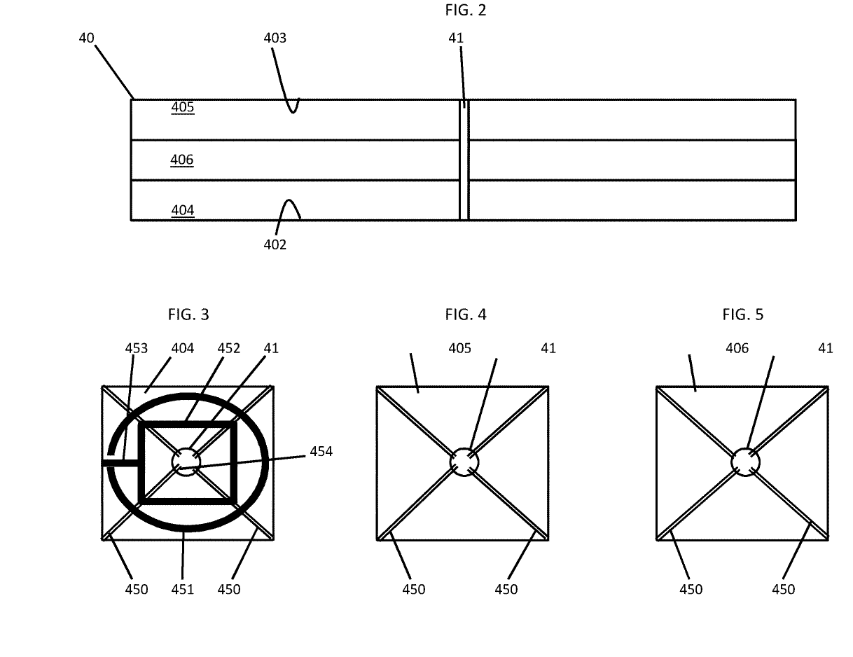

With continued reference to FIG. 1 and with additional reference to FIG. 2, the sensor body 40 may be provided as a generally planar interposing element that is disposable between the upper surface 202 of the lower cell 20 and the base surface 301 of the upper cell 30. The sensor body 40 may be formed of any dielectric or electrically insulating material and may include a body portion 401, a lower surface 402 that is disposable in contact with the upper surface 202 and an upper surface 403 that is disposable in contact with the base surface 301. The body portion 401 may be provided in multiple layers such as, for example, a lower layer 404, which includes the lower surface 402, an upper layer 405, which includes the upper surface 403, and an intermediate layer 406, which is interposed between the lower layer 404 and the upper layer 405.

With reference to FIGS. 3-5, the lower layer 404, the upper layer 405 and the intermediate layer 406 may each include various types of the sensor components 45. For example, as shown in FIG. 3, the lower layer 404 may include multiple electrodes 450 disposed proximate to the nano-pore 41, a ground element 451 that is disposed around the nano-pore 41, a processing or chip element 452 to which the multiple electrodes 450 and the ground element 451 are respectively attached and wiring 453. The multiple electrodes 450 may be provided as four individual electrodes 450 disposed proximate to the nano-pore 41. Each individual electrode 450 can have a contact lead that coats an interior of the nano-pore 41 or a tap 454 that locally penetrates into the nano-pore 41. The ground element 451 may be provided as a copper ground ring that extends around the nano-pore 41. The processing or chip element 452 may be provided with multiple electrical elements that are electrically communicative with the multiple, individual electrodes 450 and the ground element 451 to sense characteristics of the components of the strands of the DNA. The sensed characteristics of the components of the strands of the DNA may be output to an external device by way of the wiring 453.

As shown in FIGS. 4 and 5, the upper layer 405 and the intermediate layer 406 may also include respective sets of multiple electrodes 450 that are proximate to the nano-pore 41. In each case, the multiple electrodes 450 may be coupled to and electrically communicative with the processing or chip element 452. Thus, as the strands of DNA pass through the nano-pore 41, the processing or chip element 452 has a number of opportunities to redundantly identify and analyze the characteristics of the DNA strands (e.g., the sequences of bases of the strands of the DNA) based on the readings generated by the multiple electrodes 450 in each of the lower layer 404, the upper layer 405 and the intermediate layer 406.

The lower cell 20, the upper cell 30 and the sensor body 40 may be connected or secured together by various methods of attachment. As shown in FIG. 1, these may include mechanical connections such as screws or nut and bolt combinations. Alternatively, the lower cell 20, the upper cell 30 and the sensor body 40 can be connected by adhesion, metallurgical bonds, mechanical fasteners, etc.

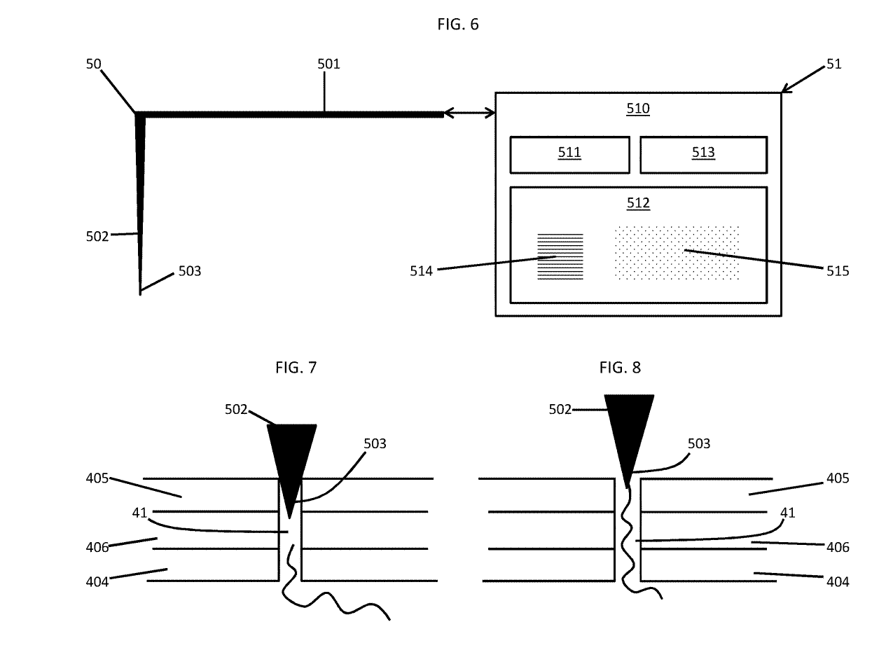

With reference back to FIG. 1 and with additional reference to FIG. 6, the SPM tip 50 is a component of an SPM apparatus 51 that includes a controller 510 and the SPM tip 50. As shown in FIGS. 1 and 6, the SPM tip 50 includes an elongate cantilever beam 501, which is coupled to the controller 510, and a tapered tip 502. The tapered tip 502 is orthogonally coupled to a distal end of the cantilever beam 501 such that a longitudinal axis of the tapered tip 502 may be transversely oriented relative to a longitudinal axis of the cantilever beam 501. A distal end of the tapered tip 502 may also include or be doped with a chemical signature or additive 503 that has an affinity with the strands of DNA. Alternatively, the tapered tip 502 may also electrically or magnetically attract the strands of DNA.

The controller 510 may include a processing unit 511, a memory unit 512 and a servo control unit 513 that controls various movements of the SPM tip 50 in accordance with control commands generated by the processing unit 511. The memory unit 512 includes multiple types of storage capacity including read-only and random-access memory and has executable instructions 514 stored thereon along with a topographic map 515 of the sensor body 40. The topographic map 515 may be provided as a top down image of the sensor body 40 that could be generated by the SPM tip 50 or by an imaging element. In any case, the topographic map 515 shows a location of the nano-pore 41 relative to the SPM tip 50 at any given time.

Using information provided by the topographic map 515 and based on control algorithms provided by the executable instructions 514, the processing unit 511 generates control commands that are issued to the SPM tip 50 by the servo control unit 513. In accordance with embodiments, these control commands cause the SPM tip 50 to be initially positioned such that the tapered tip 502 is within the interior 31 of the upper cell 30 and over the nano-pore 41. Next, with reference to FIG. 7, the control commands cause the SPM tip 50 to be lowered until the tapered tip 502 penetrates into the nano-pore 41.

As shown in FIG. 7, the lowering continues until the tapering of the tapered tip 502 causes the tapered tip 502 to contact an upper edge of the nano-aperture 411 (see FIG. 1) with the distal end of the tapered tip 502 and the chemical additive thereof disposed within the nano-pore 41. At this point, due to the Brownian motion of the strands of DNA within the interior 31 of the lower cell 30 and the affinity of the strands of DNA to the chemical additive of the tapered tip 502, the tapered tip 502 will make contact with and thus connect with at least one strand of DNA.

With reference to FIG. 8, the contact and connection between the strand of DNA and the tapered tip 502 may be recognized chemically or electro-mechanically by the processing unit 511. The processing unit 511 will then generate additional control commands that are issued to the SPM tip 50 by the servo control unit 513 that will cause the SPM tip 50 to move upwardly such that the tapered tip 502 draws the strand of DNA into and through the nano-pore 41. As such drawing occurs, the multiple electrodes 450 (see FIGS. 3-5) in the lower, upper and intermediate layers 404, 405 and 406 will sense and identify the bases of the strand of DNA.

Once the strand of DNA is completely drawn through the nano-pore 41, the SPM tip 50 may be deactivated such that the strand of DNA is released into fluid of the upper cell 30. At this point, the SPM tip 50 can be reactivated to draw another strand of DNA into and through the nano-pore 41. This process can be repeated until a predefined number of readings have been generated or until a number of the strands of DNA within the lower cell 20 drops below a predefined lower limit.

In accordance with embodiments, the distance through which the tapered tip 502 penetrates the nano-pore 41 is based on the angle of the tapering thereof and take into account an amount of chemical additive needed to reliably attract the strand of DNA as well as the range of such attraction. In accordance with alternative embodiments, however, it is to be understood that the tapered tip 502 may extend through an entirety of the nano-pore 41 and into the interior 31 of the lower cell 30 and need not be tapered as long as the positioning of the SPM tip 50 can be reliably controlled.

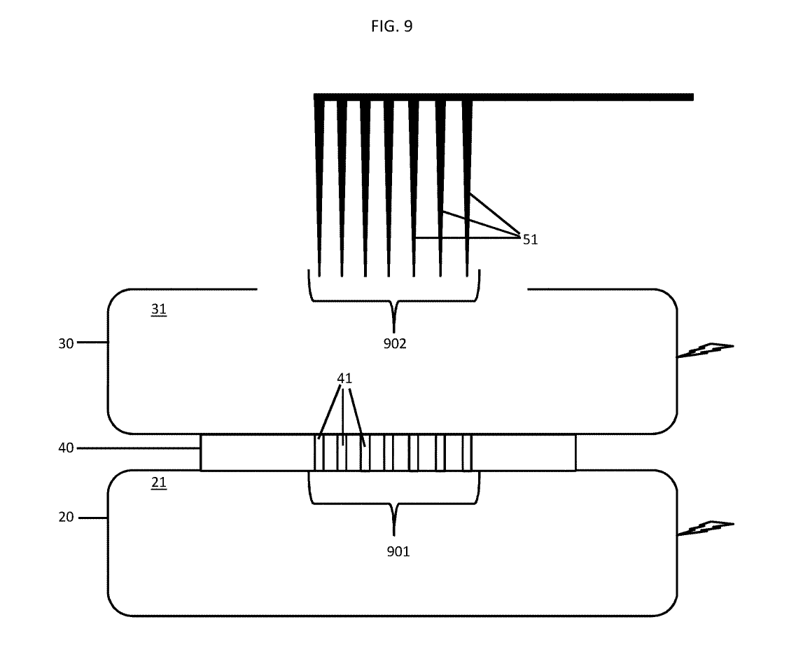

With reference to FIG. 9 and, in accordance with further embodiments, the nano-pore 41 may be provided as a plurality of nano-pores 41 and the SPM tip 50 may be provided as one or more SPM tips 51 that are configured to draw a molecule through each of the plurality of nano-pores 41. In these embodiments, the plurality of the nano-pores 41 may be provided in a matrix-like formation 901 that extends along and within a plane of the sensor body 40. Here, the topographic map 515 (see FIG. 6) correspondingly illustrates the plurality of the nano-pores 41 and provides the controller 510 with the information needed to control the SPM tip 50 (or the one or more SPM tips 50) relative to each of the plurality of the nano-pores 41. Thus, where a single SPM tip 50 is used with the plurality of the nano-pores 41, the controller 510 might control the single SPM tip 50 to visit each of the plurality of the nano-pores 41 by row and column. By contrast, where a column of SPM tips 50 is used with the plurality of the nano-pores 41, the controller 510 might control the column of SPM tips 50 to visit corresponding columns of nano-pores 41 by row. Moreover, where a plurality of SPM tips 50 is arranged in a matrix-like formation 902 that is similar to the matrix-like formation of the plurality of nano-pores 41, the plurality of SPM tips 50 may be maneuvered together to visit corresponding ones of the nano-pores 41 at a same time.

In any case, each one of the plurality of the nano-pores 41 may be provided with multiple electrodes 450 at each of the lower, upper and intermediate levels 404, 405 and 406 of the sensor body 40. Thus, readings can be independently generated at each one of the plurality of nano-pores 41.

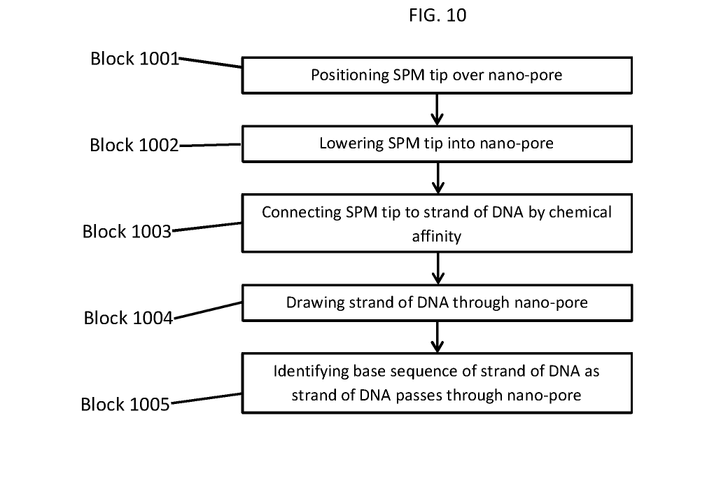

With reference to FIG. 10, a method of operating the wet cell apparatus 10 that is described herein is provided and may be executed generally by the controller 510 in an automatic mode or in a manual mode under the control of an operator. In any case, the method includes controlling a scanning probe microscope (SPM) tip to be positioned over the nano-pore 41 (block 1001) and to be lowered into the nano-pore 41 (block 1002), to connect with the strand of DNA by chemical affinity (block 1003) and to then draw the strand of DNA from the interior 21 of the lower cell 20 into the interior 31 of the upper cell 30 through the nano-pore 41 (block 1004). The method further includes identifying bases of the strand of DNA as the strand of DNA passes through the nano-pore 41 and thus interacts with the sensing components 45 of the sensor body 40 (block 1005).

The method as described herein can be modified for various numbers of nano-pores 41 and correspondingly varied numbers of SPM tips 50. The method may be repeated multiple times for each one of the nano-pores 41 and each one of the SPM tips 50 until a given number of readings are generated or until a number of strands of DNA in the interior 21 of the lower cell 20 drops below a predefined lower limit.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed is:

1. A wet cell apparatus, comprising:
a sensor body defining a nano-pore by which respective cell interiors are fluidly communicative; and
a scanning probe microscope (SPM) tip configured to draw a molecule through the nano-pore from one of the respective cell interiors whereby sensing components of the sensor body identify molecule components as the molecule passes through the nano-pore.

2. The wet cell apparatus according to claim 1, wherein the molecule comprises a DNA strand, an RNA strand or a macro-molecule and the SPM tip has a chemical affinity with the molecule.

3. The wet cell apparatus according to claim 1, wherein the sensing components identify bases of the molecule.

4. The wet cell apparatus according to claim 1, wherein the nano-pore is provided as a plurality of nano-pores and the SPM tip is provided as one or more SPM tips that are configured to draw a molecule through each of the plurality of nano-pores.

5. The wet cell apparatus according to claim 1, wherein the sensor body comprises a multi-level body formed of dielectric material.

6. The wet cell apparatus according to claim 1, wherein the sensing components comprise:
electrodes disposed proximate to the nano-pore;
a ground element disposed about the nano-pore; and
electrical elements electrically communicative with the electrodes and the ground ring to sense characteristics of the molecule components.

7. The wet cell apparatus according to claim 6, wherein the electrodes comprise multiple electrodes at multiple levels of the sensor body and the ground element comprises a ground ring surrounding the nano-pore.

8. The wet cell apparatus according to claim 6, wherein the electrodes comprise taps extending into the nano-pore.

9. The wet cell apparatus according to claim 1, wherein the SPM tip comprises:
a cantilever beam; and
a tapered tip, which is orthogonally coupled to a distal end of the cantilever beam and which has an affinity with the molecule.

10. The wet cell apparatus according to claim 1, further comprising a controller coupled to the SPM tip and configured to control a position of the SPM tip.

11. A wet cell apparatus, comprising:
lower and upper cells;
a sensor body formed to define a nano-pore disposed to permit fluid communication between respective interiors of the lower and upper cells; and
a scanning probe microscope (SPM) tip configured to draw a molecule from the interior of the lower cell into the interior of the upper cell through the nano-pore,
the sensor body comprising sensing components configured to identify components of the molecule as the molecule passes through the nano-pore.

12. The wet cell apparatus according to claim 11, wherein the molecule comprises a DNA strand, an RNA strand or a macro-molecule and the SPM tip has a chemical affinity with the molecule.

13. The wet cell apparatus according to claim 11, wherein the sensing components identify bases of the molecule.

14. The wet cell apparatus according to claim 11, wherein the nano-pore is provided as a plurality of nano-pores and the SPM tip is provided as one or more SPM tips that are configured to draw a molecule through each of the plurality of nano-pores.

15. The wet cell apparatus according to claim 11, wherein the sensor body comprises a multi-level body formed of dielectric material.

16. The wet cell apparatus according to claim 11, wherein the sensing components comprise:
electrodes disposed proximate to the nano-pore;
a ground element disposed about the nano-pore; and
electrical elements electrically communicative with the electrodes and the ground ring to sense characteristics of the components of the molecule.

17. The wet cell apparatus according to claim 16, wherein the electrodes comprise multiple electrodes at multiple levels of the sensor body and the ground element comprises a ground ring surrounding the nano-pore.

18. The wet cell apparatus according to claim 6, wherein the electrodes comprise taps extending into the nano-pore.

19. The wet cell apparatus according to claim 11, wherein the SPM tip comprises:
a cantilever beam; and
a tapered tip, which is orthogonally coupled to a distal end of the cantilever beam and which has an affinity with the molecule.

20. A method of operating a wet cell apparatus comprising lower and upper cells disposed on either side of a sensor body comprising sensing components and defining a nano-pore with respective interiors of the lower and upper cells charged with a fluid and the interior of the lower cell initially charged with molecules, the method comprising:
controlling a scanning probe microscope (SPM) tip to draw at least one of the molecules from the interior of the lower cell into the interior of the upper cell through the nano-pore; and
identifying components of the molecule as the molecule passes through the nano-pore and interacts with the sensing components.
